(19) United States
(12) Plant Patent Application Publication
(10) Pub. No.: US 2023/0270028 P1
(43) Pub. Date: Aug. 24, 2023

(54) Blueberry Plant Variety Named Bluecsol9

(50) ;

(71) Applicant: CAMPOSOL S.A., Lima (PE)

(72) Inventors: Kimberlayn Maria Apsara Sanabria Aguilar, Lima (PE); Milagros Janet Granda Mogollon De Montoya, Lima (PE); Luis José Montgomery Taboada, Lima (PE); Jorge David Romero Pajares, Lima (PE)

(73) Assignee: CAMPOSOL S.A., Lima (PE)

(21) Appl. No.: 17/803,892

(22) Filed: Jan. 11, 2023

(30) Foreign Application Priority Data
Jan. 14, 2022 (PE) 73-2022

(51) Int. Cl. A01H 6/36

(52) U.S. Cl. PLT/157

(57) ABSTRACT

A new and distinct evergreen blueberry variety with zero chill requirement named BLUECSOL9, characterized by the following combination of traits: medium to strong plant vigor, semi-upright growth habit, produces large fruit-size, strong bloom intensity, oblata fruit shaped, high sweetness and medium acidity and very firm fruit. Latin name of the genus and species: Vaccinium corymbosum L.

Latin name: Vaccinium corymbosum L.

Variety denomination: Blueberry plant named BLUECSOL9

### CROSS REFERENCE TO RELATED APPLICATIONS

This application claims priority to a Peruvian Breeders Rights Application No. 73-2022 filed on Jan. 14, 2021, the entire contents of which is incorporated by reference herein.

### BACKGROUND OF THE INVENTION

The present invention relates to a new Vaccinium corymbosum L. plant, which was selected from open pollination between Biloxi (Vaccinium corymbosum L., public plant) as the female (seed) parent and pollen from an unidentified variety (Vaccinium corymbosum L.). Biloxis' berries were collected from different fields of the CAMPOSOL S. A company, located in Viru, La Libertad, Peru at the beginnings of 2016 to extract the seeds. 11805 seedlings at high density were planted under field conditions in December of the same year.

The seedlings were evaluated during 3 growing season (2017, 2018 and 2019). The breeding method used was phenotypic recurrent selection. In December 2019 one of the seedlings, BLUECSOL 9, was isolated due to its good quality fruit, long postharvest life, and high yield potential, and was asexually propagated by softwood cuttings to confirm the distinctness, and stability of the characteristics observed. In an experimental test plot of the Blueberry Breeding Program of the CAMPOSOL S. A company, located in Viru, La Libertad, Peru, 5 rooted cuttings were planted. From December 2020 until the present, said test plot has shown that the unique features of this new Vaccinium variety are stable and reproduce true to type in successive generations of asexual propagation. In addition, BLUECSOL9 was subsequently propagated by softwood cutting and tissue culture and an additional plot, with 100 plants were planted in December 2021 in the same location. This new plot has maintained up to the present the distinctness, and stability characteristics of the new variety of Vaccinium corymbosum L.

### SUMMARY OF THE VARIETY

The following is summary of the description of the new and distinct variety of blueberry “BLUECSOL9” was selected in Viru, La Libertad, Peru in December 2019. BLUECSOL9 is an evergreen blueberry variety with zero chill requirement. The following are the most distinguishing traits of this new variety: medium to strong plant vigor, semi-upright growth habit, ovate leaf shape, globose corolla shape, oblata fruit shaped, large fruit-size, strong bloom intensity, high sweetness and medium acidity and very firm fruit.

BLUECSOL9 was selected as an early variety, with high yield potential, excellent fruit quality and post-harvest life.

### BRIEF DESCRIPTION OF THE DRAWINGS

The accompanying photographs show typical bush, leaves, flower, and fruit characteristics of the new Vaccinium corymbosum L.BLUECSOL9. Colors shown are as accurate as can be reasonably reproduced by the photographic means. Photographs were taken of 2 and 4-year-old plants grown in fields of the CAMPOSOL S. A. located in Viru, La Libertad, Peru.

FIG. 1—Shows typical bush with semi-upright growth habit on 4-year-old plant of the new variety BLUECSOL9.

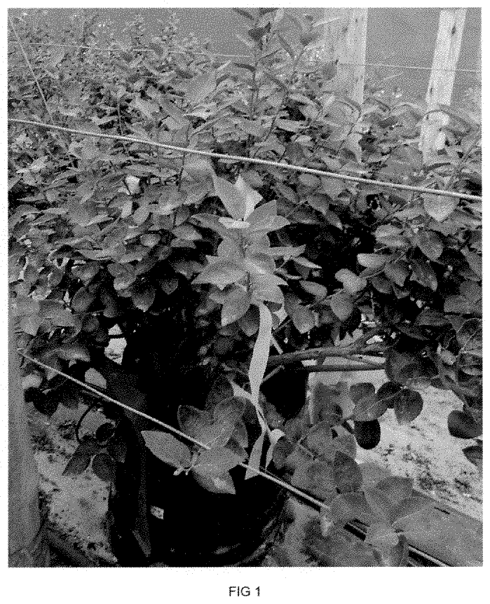

FIG. 2—Shows several clusters of opening flowers on 4-year-old plant of the new variety BLUECSOL9.

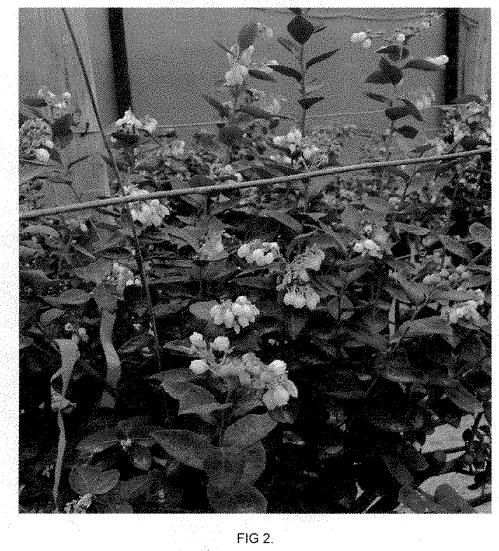

FIG. 3—Shows the size and shape of the flower corolla on 2-year-old plant of the new variety BLUECSOL9.

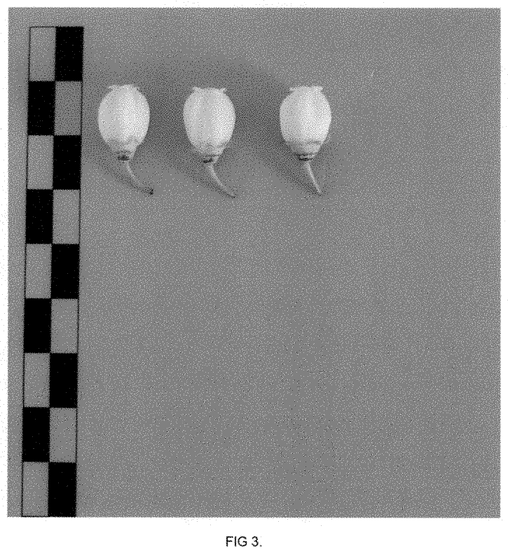

FIG. 4—Shows the upper sides and shape of complete leaves on 2-year-old plant of the new variety BLUECSOL9.

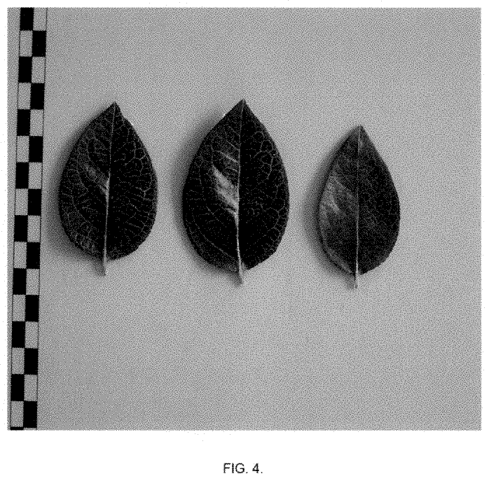

FIG. 5.—Shows the underside of a complete leave on 2-year-old plant of the new variety BLUECSOL9.

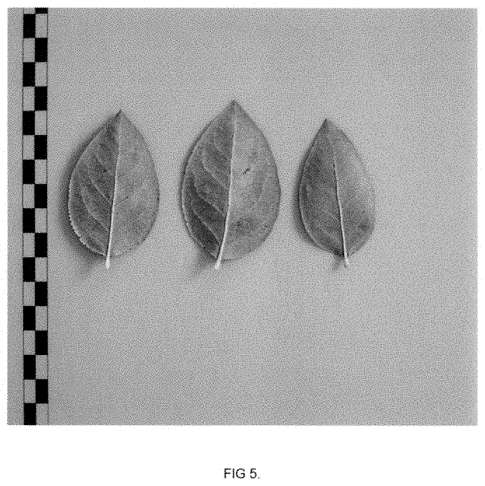

FIG. 6.—Shows the size and shape of the fruits on 2-year-old plant of the new variety BLUECSOL9.

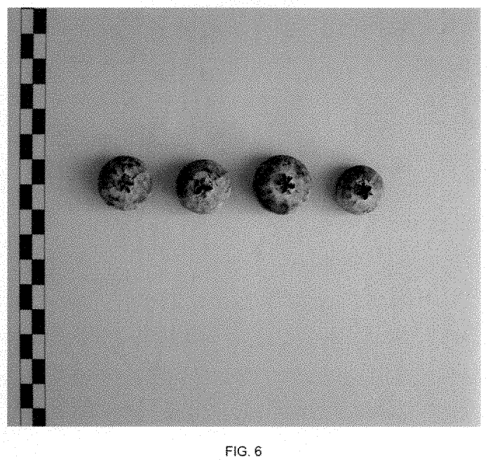

### DETAILED BOTANICAL DESCRIPTION

The following botanical description detailed forth distinctive traits of BLUECSOL9. The data were collected from clones stablished in an experimental test plot in Peru on 2-year-old plants. Color descriptions are based on the Pantone® Munsell Plant Tissue Color Book scale.
Classification:
   Family.—Ericaceae.
   Species.—Vaccinium corymbosum L.
   Common name.—Southern Highbush Blueberry.
   Cultivar name.—BLUECSOL9.
   Parentage: Female parent.—Biloxi (Unpatented).
   Male parent.—Unidentified variety.
Plant:
   Vigor.—Medium to strong.
   Growth habit.—Semi-upright.
   Height.—Mean of 150 cm.
   Width.—Mean of 130 cm.
   Internode length.—Mean of 21.56 mm.
   Evergreenness.—Evergreen.
   Chilling requirements.—0 hours below 7° C.
   Ease of propagation.—BLUECSOL9 shows a high-rate multiplication when it was propagated by tissue cultured.
   Color of 1-year-old rough bark observed November 8.—Brown Pantone® 5GY 6/4.
Leaves:
   Length.—Mean of 55 mm.
   Width.—Mean of 35 mm.
   Length/width ratio.—1.57.
   Shape.—Ovate.
   Margin.—Serrate.
   Color on upper side of old leaves.—Green Pantone® 5GY 3/4.
   Color on upper side of young leaves.—Green Pantone® 5GY 4/6.
Flowers:
   Flower cluster.—Medium.
   Flower fragrance.—Little or none.
   Inflorescence length.—Mean of 42 mm.
   Size of corolla tube.—Mean of 12 mm (from pedicel attachment point to corolla tip excluding the pedicel).
   Corolla shape.—Globose.
   Length of corolla tube.—Mean of 12.3 mm.
   Style length.—Top of ovary to stigma tip. 8 mm.
   Flower cluster.—Medium.
Fruits:
   Diameter of calyx aperture on mature berry.—Mean of 4.5 mm.
   Diameter o depth of calyx on mature berry.—Mean of 2.7 mm.
   Detachment force for ripe berries (easy, medium, hard).—Easy.
   Berries per cluster.—About 8 to 12.
   Width.—About 16 to 20 mm.
   Weight.—Mean of 2.5 g per berry.
   Shape.—Oblate.
   Intensity of fruit bloom.—Strong.
   Pedicel scar.—Mean of 2 mm.
   Color of unripe fruit.—Light green Pantone® 2.5GY 6/8.
   Color of fruit flesh.—Pantone® 2.5GY 7/4.
   Fruit firmness.—Very firm.
   Fruit acidity.—Medium.
   Fruit sweetness.—High.
   Market use of fruit.—Fresh market.
Seeds:
   Color.—Brown Pantone® 7.5YR 4/4.
   Seeds per berry.—Mean of 10 seeds.
Reproductive organs:
   Pollen abundance.—Abundant.
   Self-compatibility.—High degree of self-compatibility.
   Disease, insects, and mites.—No sensitivity to any disease and pest has been observed for BLUECSOL9.

### COMPARISON TO CLOSEST VARIETY

BLUECSOL9 is distinguished of Biloxi variety in the following characteristics:

Color on upper side of old leaves in plants of Biloxi (Unpatented) variety is green Pantone® 2.5G 3/2, whereas the color on upper side in leaves of BLUECSOL9 is green Pantone® 5GY 3/4.

The length of the inflorescence in Biloxi (Unpatented) is medium and in BLUECSOL9 is short.

Fruit size of Biloxi (Unpatented) is medium and shows high acidity, whereas in BLUECSOL9 fruit size large fruits and medium acidity.

The diameter of depth of calyx on mature berry in Biloxi (Unpatented) is shorter than BLUECSOL9.

Intensity of fruit bloom in in Biloxi (Unpatented) is weaker than BLUECSOL9.

What is claimed is:

1. A new and distinct blueberry plant named BLUECSOL9 substantially as illustrated and described herein.
